(19) United States
(12) Patent Application Publication
(10) Pub. No.: US 2019/0343953 A1
(43) Pub. Date: Nov. 14, 2019

(54) ANTI-CANCER TREATMENTS WITH AN ANTI-MUC1 ANTIBODY AND AN ERBB INHIBITOR

(71) Applicant: Glycotope GmbH, Berlin (DE)

(72) Inventors: Steffen GOLETZ, Berlin (DE); Johanna RÜHMANN, Berlin (DE); Beate HABEL, Berlin (DE); Felix HART, Berlin (DE)

(21) Appl. No.: 16/478,297

(22) PCT Filed: Jan. 24, 2018
(86) PCT No.: PCT/EP2018/051652

§ 371 Date: Jul. 16, 2019

(30) Foreign Application Priority Data
Jan. 27, 2017 (LU) LU100026

(51) Int. Cl. A61K 39/395; A61K 31/40; A61K 31/517; A61P 35/00

(52) U.S. Cl. CPC A61K 39/3955; A61K 31/40; A61K 2300/00; A61P 35/00; A61K 31/517

(57) ABSTRACT

The present invention pertains to the field of cancer therapy using anti-cancer antibodies. The medical use of anti-MUC1 antibodies in combination with inhibitors of the ErbB receptor family is provided which show synergistic anti-cancer efficacy.

### FIELD OF THE INVENTION

The present invention pertains to a novel combination therapy against cancer. The combination of an inhibitor of a receptor of the ErbB family such as EGFR and an antibody against MUC1 results in an improved effect in cancer treatment. The present invention therefore provides the use of the two therapeutic agents in combination for the treatment of cancer.

### BACKGROUND OF THE INVENTION

Antibodies are widely used agents in the field of medicine and research. In medicine, they find application in many different fields, in particular as therapeutic agents in the treatment and prophylaxis of a variety of diseases, in particular neoplastic diseases such as cancer. However, therapeutic results obtained by antibody therapy of cancer patients are highly variable. A significant percentage of the therapies using anti-cancer antibodies shows no or only a small alleviation of the disease and sometimes are limited to specific patient groups.

An interesting and important group of antibodies are those directed against mucin proteins. Mucins are a family of high molecular weight, heavily glycosylated proteins produced by many epithelial tissues in vertebrates. They can be subdivided into mucin proteins which are membrane-bound due to the presence of a hydrophobic membrane-spanning domain that favors retention in the plasma membrane, and mucins which are secreted onto mucosal surfaces or secreted to become a component of saliva. The human mucin protein family consists of at least the family members MUC1, MUC2, MUC3A, MUC3B, MUC4, MUC5AC, MUC5B, MUC6, MUC7, MUC8, MUC12, MUC13, MUC15, MUC16, MUC17, MUC19, and MUC20; wherein MUC1, MUC3A (isoform 1), MUC3B, MUC4 and MUC16 are membrane bound.

Increased mucin production occurs in many adenocarcinomas, including cancer of the pancreas, lung, breast, ovary, colon, etc. Mucins are also overexpressed in lung diseases such as asthma, bronchitis, chronic obstructive pulmonary disease or cystic fibrosis. Two membrane mucins, MUC1 and MUC4 have been extensively studied in relation to their pathological implication in the disease process. Moreover, mucins are also being investigated for their potential as diagnostic markers.

Several antibodies directed against mucin proteins, in particular MUC1, are known in the art. Some of them are already approved for medical applications.

Another established cancer target are the receptors of the ErbB family, in particular EGFR (epidermal growth factor receptor) and HER2. The ErbB receptors are receptor tyrosine kinases which are anchored in the plasma membrane. Binding of the ligands epidermal growth factor (EGF) or transforming growth factor alpha (TGFα) to the extracellular domain of an ErbB receptor results in (homo- or hetero-) dimerization of the receptor and stimulation of its intracellular protein-tyrosine kinase activity. The signal transduction cascades initiated by the active receptor dimer control cell migration, adhesion, and proliferation. The human ErbB receptor proteins are thought to be useful targets for therapy against cancers expressing said proteins. For example, EGFR is over-expressed in several cancers, including but not limited to colorectal cancer, lung cancer, pancreatic cancer and head-and-neck cancer. Mutations, amplifications or misregulations of EGFR or family members are implicated in about 30% of all epithelial cancers and are associated with a poor prognosis.

Inhibitors of ErbB receptors including small molecule kinase inhibitors and antibodies are established anti-cancer drugs. The anti-ErbB antibodies are effective in cancer treatment because they are able to inhibit ErbB signaling. They bind to the extracellular domain of the receptor and prevent binding of the natural activating ligands such as EGF and TGFα, thereby inhibiting dimerization and activation of the receptor and its downstream signaling cascade. The kinase inhibitors prevent the ErbB receptors from phosphorylating each other after dimerization. Phosphorylation of the receptors is important for binding of the downstream signaling partners.

It is to be noted that these mechanisms of action are only relevant for tumors which depend on the activation of ErbB receptors for proliferation. Especially in colorectal cancer, a large portion of the tumors, however, comprise a mutation in the Kirsten Ras gene (KRAS), rendering the K-Ras protein constantly active. K-Ras is an important member of the downstream signaling cascade of EGFR and an inhibition of EGFR signaling will generally have no effect on tumors wherein K-Ras is constantly active. Because of this, some drugs targeting ErbB receptors are only approved for the treatment of KRAS wild-type metastatic colorectal cancer.

In view of the huge variety of different cancers and the known limitations of existing treatments, there is a constant need for further cancer treatments with higher efficacy and less adverse side effects.

### SUMMARY OF THE INVENTION

Treatment of tumors expressing an ErbB receptor such as EGFR or HER2 with an inhibitor of the respective receptor showed synergistic effects in combination with an antibody against MUC1. The receptor inhibitor induced increased expression of MUC1, which could effectively be targeted by anti-MUC1 antibodies. The higher MUC1 level on the tumor cells resulted in an increased efficacy of the anti-MUC1 antibody. Optimal results were obtained when the anti-MUC1 antibody was added some time after the receptor inhibitor, in particular about 1 to 6 days thereafter.

In a first aspect, the present invention provides an antibody against MUC1 (anti-MUC1 antibody) for use in the treatment of cancer in combination with an inhibitor of a receptor of the ErbB family (ErbB inhibitor).

In a second aspect, the present invention provides an inhibitor of a receptor of the ErbB family for use in the treatment of cancer in combination with an antibody against MUC1.

In addition, the present invention likewise provides methods of treatment in accordance with the first and/or second aspect. In particular, the present invention provides a method of treatment of cancer, comprising administering to the patient in need thereof an inhibitor of a receptor of the ErbB family and an antibody against MUC1. All the embodiments and features described herein for the first and second aspect of the invention also likewise apply to the methods of treatment according to the invention.

The above aspects can be combined. Other objects, features, advantages and aspects of the present invention will become apparent to those skilled in the art from the following description and appended claims. It should be understood, however, that the following description, appended claims, and specific examples, which indicate preferred embodiments of the application, are given by way of illustration only. Various changes and modifications within the spirit and scope of the disclosed invention will become readily apparent to those skilled in the art from reading the following.

### DEFINITIONS

As used herein, the following expressions are generally intended to preferably have the meanings as set forth below, except to the extent that the context in which they are used indicates otherwise.

The expression “comprise”, as used herein, besides its literal meaning also includes and specifically refers to the expressions “consist essentially of” and “consist of”. Thus, the expression “comprise” refers to embodiments wherein the subject-matter which “comprises” specifically listed elements may and/or indeed does encompass further elements as well as embodiments wherein the subject-matter which “comprises” specifically listed elements does not comprise further elements. Likewise, the expression “have” is to be understood as the expression “comprise”, also including and specifically referring to the expressions “consist essentially of” and “consist of”.

The term “antibody” in particular refers to a protein comprising at least two heavy chains and two light chains connected by disulfide bonds. Each heavy chain is comprised of a heavy chain variable region (VH) and a heavy chain constant region (CH). Each light chain is comprised of a light chain variable region (VL) and a light chain constant region (CL). The heavy chain-constant region comprises three or—in the case of antibodies of the IgM- or IgE-type—four heavy chain-constant domains (CH1, CH2, CH3 and CH4) wherein the first constant domain CH1 is adjacent to the variable region and may be connected to the second constant domain CH2 by a hinge region. The light chain-constant region consists only of one constant domain. The variable regions can be further subdivided into regions of hypervariability, termed complementarity determining regions (CDRs), interspersed with regions that are more conserved, termed framework regions (FR), wherein each variable region comprises three CDRs and four FRs. The variable regions of the heavy and light chains contain a binding domain that interacts with an antigen. The heavy chain constant regions may be of any type such as γ-, δ-, α-, μ- or ε-type heavy chains. Preferably, the heavy chain of the antibody is a γ-chain. Furthermore, the light chain constant region may also be of any type such as κ- or λ-type light chains. Preferably, the light chain of the antibody is a κ-chain. The constant regions of the antibodies may mediate the binding of the immunoglobulin to host tissues or factors, including various cells of the immune system (e.g., effector cells) and the first component (C1q) of the classical complement system. The antibody can be e.g. a humanized, human or chimeric antibody. The antibody may be capable of inducing ADCC.

The antigen-binding portion of an antibody usually refers to full length or one or more fragments of an antibody that retain the ability to specifically bind to an antigen. It has been shown that the antigen-binding function of an antibody can be performed by fragments of a full-length antibody. Examples of binding fragments of an antibody include a Fab fragment, a monovalent fragment consisting of the V_{L}, V_{H}, C_{L} and C_{H1} domains; a F(ab)₂ fragment, a bivalent fragment comprising two Fab fragments, each of which binds to the same antigen, linked by a disulfide bridge at the hinge region; a Fd fragment consisting of the V_{H} and C_{H1} domains; a Fv fragment consisting of the V_{L} and V_{H} domains of a single arm of an antibody; and a dAb fragment, which consists of a V_{H} domain.

The “Fab part” of an antibody in particular refers to a part of the antibody comprising the heavy and light chain variable regions (V_{H} and V_{L}) and the first domains of the heavy and light chain constant regions (C_{H1} and C_{L}). In cases where the antibody does not comprise all of these regions, then the term “Fab part” only refers to those of the regions V_{H}, V_{L}, C_{H1} and C_{L} which are present in the antibody. Preferably, “Fab part” refers to that part of an antibody corresponding to the fragment obtained by digesting a natural antibody with papain which contains the antigen binding activity of the antibody. In particular, the Fab part of an antibody encompasses the antigen binding site or antigen binding ability thereof. Preferably, the Fab part comprises at least the V_{H} region of the antibody.

The “Fc part” of an antibody in particular refers to a part of the antibody comprising the heavy chain constant regions 2, 3 and—where applicable—4 (C_{H2}, C_{H3} and C_{H4}). In particular, the Fc part comprises two of each of these regions. In cases where the antibody does not comprise all of these regions, then the term “Fc part” only refers to those of the regions C_{H2}, C_{H3} and C_{H4} which are present in the antibody. Preferably, the Fc part comprises at least the C_{H2} region of the antibody. Preferably, “Fc part” refers to that part of an antibody corresponding to the fragment obtained by digesting a natural antibody with papain which does not contain the antigen binding activity of the antibody. In particular, the Fc part of an antibody is capable of binding to the Fc receptor and thus, e.g. comprises an Fc receptor binding site or an Fc receptor binding ability.

According to the present invention, the term “chimeric antibody” in particular refers to an antibody wherein the constant regions are derived from a human antibody or a human antibody consensus sequence, and wherein at least one and preferably both variable regions are derived from a non-human antibody, e.g. from a rodent antibody such as a mouse antibody.

According to the present invention, the term “humanized antibody” in particular refers to a non-human antibody comprising human constant regions and variable regions which amino acid sequences are modified so as to reduce the immunogenicity of the antibody when administered to the human body. An exemplary method for constructing humanized antibodies is CDR grafting, wherein the CDRs or the specificity determining residues (SDRs) of a non-human antibody are combined with human-derived framework regions. Optionally, some residues of the human framework regions may be backmutated towards the residues of the parent non-human antibody, e.g. for increasing or restoring the antigen binding affinity. Other humanization methods include, for example, resurfacing, superhumanization, and human string content optimization. In the resurfacing methods, only those residues of the non-human framework regions which are positioned at the surface of the antibody are replaced by residues present in corresponding human antibody sequences at said position. Superhumanization essentially corresponds to CDR grafting. However, while during CDR grafting the human framework regions are normally chosen based on their homology to the non-human framework regions, in superhumanization it is the similarity of the CDRs on the basis of which the human framework regions are chosen. In the human string content optimization the differences of the non-human antibody sequence to the human germline sequences is scored and then the antibody is mutated to minimize said score. Furthermore, humanized antibodies can also be obtained by empirical methods wherein large libraries of human framework regions or human antibodies are used to generate multiple antibody humanized candidates and then the most promising candidate is determined by screening methods. Also with the above-described rational approaches several humanized antibody candidates can be generated and then screened, for example for their antigen binding.

The term “human antibody”, as used herein, is intended to include antibodies having variable regions in which both the framework and CDR regions are derived from sequences of human origin.

The term “antibody”, as used herein, refers in certain embodiments to a population of antibodies of the same kind. In particular, all antibodies of the population of the antibody exhibit the features used for defining the antibody. In certain embodiments, all antibodies in the population of the antibody have the same amino acid sequence. Reference to a specific kind of antibody, such as an anti-MUC1 antibody, in particular refers to a population of this kind of antibody.

The term “antibody” as used herein also includes fragments and derivatives of said antibody. A “fragment or derivative” of an antibody in particular is a protein or glycoprotein which is derived from said antibody and is capable of binding to the same antigen, in particular to the same epitope as the antibody. Thus, a fragment or derivative of an antibody herein generally refers to a functional fragment or derivative. In particularly preferred embodiments, the fragment or derivative of an antibody comprises a heavy chain variable region. It has been shown that the antigen-binding function of an antibody can be performed by fragments of a full-length antibody or derivatives thereof. Examples of fragments or derivatives of an antibody include (i) Fab fragments, monovalent fragments consisting of the variable region and the first constant domain of each the heavy and the light chain; (ii) F(ab)₂ fragments, bivalent fragments comprising two Fab fragments linked by a disulfide bridge at the hinge region; (iii) Fd fragments consisting of the variable region and the first constant domain CH1 of the heavy chain; (iv) Fv fragments consisting of the heavy chain and light chain variable region of a single arm of an antibody; (v) scFv fragments, Fv fragments consisting of a single polypeptide chain; (vi) (Fv)₂ fragments consisting of two Fv fragments covalently linked together; (vii) a heavy chain variable domain; and (viii) multibodies consisting of a heavy chain variable region and a light chain variable region covalently linked together in such a manner that association of the heavy chain and light chain variable regions can only occur intermolecular but not intramolecular. These antibody fragments and derivatives are obtained using conventional techniques known to those with skill in the art.

A target amino acid sequence is “derived” from or “corresponds” to a reference amino acid sequence if the target amino acid sequence shares a homology or identity over its entire length with a corresponding part of the reference amino acid sequence of at least 75%, more preferably at least 80%, at least 85%, at least 90%, at least 93%, at least 95%, at least 97%, at least 98% or at least 99%. The “corresponding part” means that, for example, framework region 1 of a heavy chain variable region (FRH1) of a target antibody corresponds to framework region 1 of the heavy chain variable region of the reference antibody. In particular embodiments, a target amino acid sequence which is “derived” from or “corresponds” to a reference amino acid sequence is 100% homologous, or in particular 100% identical, over its entire length with a corresponding part of the reference amino acid sequence. A “homology” or “identity” of an amino acid sequence or nucleotide sequence is preferably determined according to the invention over the entire length of the reference sequence or over the entire length of the corresponding part of the reference sequence which corresponds to the sequence which homology or identity is defined.

The term “antibody” as used herein also refers to multivalent and multispecific antibodies, i.e. antibody constructs which have more than two binding sites each binding to the same epitope and antibody constructs which have one or more binding sites binding to a first epitope and one or more binding sites binding to a second epitope, and optionally even further binding sites binding to further epitopes.

“Specific binding” preferably means that an agent such as an antibody binds stronger to a target such as an epitope for which it is specific compared to the binding to another target. An agent binds stronger to a first target compared to a second target if it binds to the first target with a dissociation constant (K_{d}) which is lower than the dissociation constant for the second target. Preferably the dissociation constant for the target to which the agent binds specifically is more than 100-fold, 200-fold, 500-fold or more than 1000-fold lower than the dissociation constant for the target to which the agent does not bind specifically. Furthermore, the term “specific binding” in particular indicates a binding affinity between the binding partners with an affinity constant Kₐ of at least 10⁶ M⁻¹, preferably at least 10⁷ M⁻¹, more preferably at least 10⁸ M⁻¹. An antibody specific for a certain antigen in particular refers to an antibody which is capable of binding to said antigen with an affinity having a Kₐ of at least 10⁶ M⁻¹, preferably at least 10⁷ M⁻¹, more preferably at least 10⁸ M⁻¹. For example, the term “anti-MUC1 antibody” refers to an antibody specifically binding MUC1 and preferably is capable of binding to MUC1 with an affinity having a Kₐ of at least 10⁶ M⁻¹, preferably at least 10⁷ M⁻¹, more preferably at least 10⁸ M⁻¹.

The term “PankoMab” as used herein in particular refers to an antibody having the amino acid sequences of the antibody PankoMab or of a humanized version thereof as disclosed in WO 2011/012309 A1.

According to the invention, the term “glycosylation site” in particular refers to an amino acid sequence which can specifically be recognized and glycosylated by a natural glycosylation enzyme, in particular a glycosyltransferase, preferably a naturally occurring mammalian or human glycosyltransferase. In particular, the term “glycosylation site” refers to an N-glycosylation site, comprising an asparagine residue to which the carbohydrate is or can be bound. In particular, the glycosylation site is an N-glycosylation site which has the amino acid sequence Asn-Xaa-Ser/Thr/Cys, wherein Xaa is any amino acid residue. Preferably, Xaa is not Pro.

A “relative amount of glycans” according to the invention refers to a specific percentage or percentage range of the glycans attached to the antibodies of an antibody population or in a composition comprising antibodies, respectively. In particular, the relative amount of glycans refers to a specific percentage or percentage range of all glycans comprised in the antibodies and thus, attached to the antibody polypeptide chains in an antibody population or in a composition comprising antibodies. 100% of the glycans refers to all glycans attached to the antibodies of the antibody population or in a composition comprising antibodies, respectively. In specific embodiments, only the glycans attached to specific glycosylation sites of the antibodies are considered. For example, a relative amount of glycans attached to the Fc part of an antibody only refers to those glycans which are attached to glycosylation sites in the Fc part of the antibodies of an antibody population or in a composition comprising antibodies, respectively.

For example, a relative amount of glycans carrying bisecting GlcNAc of 20% refers to an antibody population wherein 20% of all glycans attached to the glycosylation sites of the antibodies in said antibody composition comprise a bisecting GlcNAc residue while 80% of all glycans attached to the glycosylation sites of the antibodies in said antibody population do not comprise a bisecting GlcNAc residue.

An antibody having in a specific region, such as the Fc region, a relative amount of glycans carrying a specific saccharide unit or feature, such as fucose, galactose, two galactoses, bisecting GlcNAc, sialic acid or two sialic acids, of a specific percentage value or range in particular refers to a population of said antibodies all having the same amino acid sequence, wherein said percentage or percentage range of all glycans attached to said specific region of all of said antibodies of the population comprise said specific saccharide unit or fulfill the feature. The terms “carbohydrate chain”, “carbohydrate structure”, “glycan” and “glycan structure” as used herein have the same meaning and are used interchangeably.

According to the invention, the (percentage) amount of fucose in the Fc part and thus the CH2 domain of a specific antibody in particular refers to the percentage of all carbohydrate chains attached to the corresponding glycosylation site in the CH2 domain of the antibodies in the population of said specific antibody which comprise a fucose residue. Said carbohydrate chains include the carbohydrate chains attached to the glycosylation site corresponding structurally or by amino acid sequence homology to amino acid position 297 according to the Kabat numbering of the heavy chain of IgG-type antibodies. The N-linked glycosylation at Asn297 is conserved in mammalian IgGs as well as in homologous regions of other antibody isotypes. Antibodies usually comprise two heavy chains and two light chains and hence, have two glycosylation sites in their Fc part, one in each CH2 domain. For the avoidance of doubt, it is provided that it is not mandatory that both glycosylation sites in the CH2 domains of the antibody have to carry a carbohydrate chain. It is not distinguished between the two glycosylation sites in the two CH2 domains and referring to a glycosylation domain in the CH2 domain also refers to both glycosylation sites in both CH2 domains. Preferably, only fucose residues are considered which are bound via an a1,6-linkage to the GlcNAc residue at the reducing end of the carbohydrate chain. If the amount of fucose in the CH2 domain of a specific antibody species is mentioned, then only the carbohydrate chains attached to the glycosylation site of the CH2 domains of the antibody molecules of the population of said specific antibody species in a composition are considered for determining the percentage amount of fucose, i.e. the amount of carbohydrate chains carrying a fucose. Carbohydrate chains attached to glycosylation sites in the Fab part of the antibody, if present, as well as carbohydrate chains attached to other antibodies, if present in a composition together with the antibody of interest, are not considered for determining the amount of fucose in the CH2 domain of the antibody of interest. Carbohydrates attached to the Fab part and the Fc part of an antibody can be determined separately by first digesting the antibody in a Fab part and a Fc part, separating the parts from each other and individually determining the glycosylation features of each part. Likewise, the (percentage) amount of other saccharide residues or structural elements such as bisecting N-acetylglucosamine (bisGlcNAc), (at least one or at least two) galactose, (at least one or at least two) sialic acid, attached to the CH2 domain of an antibody in particular refers to the percentage of all carbohydrate chains attached to the glycosylation site in the CH2 domain of all antibodies in the population which comprise said saccharide residue or structural element.

The term “sialic acid” in particular refers to any N- or O-substituted derivatives of neuraminic acid. It may refer to both 5-N-acetylneuraminic acid and 5-N-glycolylneuraminic acid, but preferably only refers to 5-N-acetylneuraminic acid. The sialic acid, in particular the 5-N-acetylneuraminic acid preferably is attached to a carbohydrate chain via a 2,3- or 2,6-linkage. Preferably, in the antibody preparations described herein both 2,3- as well as 2,6-coupled sialic acids are present. The term “bisGlcNAc” or “bisecting GlcNAc” refers to a bisecting N-acetylglucosamine residue, i.e. a N-acetylglucosamine residue attached to the central mannose residue in complex type N-glycans.

The numbers given herein, in particular the relative amounts of a specific glycosylation property, are preferably to be understood as approximate numbers. In particular, the numbers preferably may be up to 10% higher and/or lower, in particular up to 9%, 8%, 7%, 6%, 5%, 4%, 3%, 2% or 1% higher and/or lower.

In a “conjugate” two or more compounds are linked together. In certain embodiments, at least some of the properties from each compound are retained in the conjugate. Linking may be achieved by a covalent or non-covalent bond. Preferably, the compounds of the conjugate are linked via a covalent bond. The different compounds of a conjugate may be directly bound to each other via one or more covalent bonds between atoms of the compounds. Alternatively, the compounds may be bound to each other via a chemical moiety such as a linker molecule wherein the linker is covalently attached to atoms of the compounds. If the conjugate is composed of more than two compounds, then these compounds may, for example, be linked in a chain conformation, one compound attached to the next compound, or several compounds each may be attached to one central compound.

The term “MUC1” refers to the protein MUC1 or mucin 1. In particular, it refers to the human MUC1 protein. MUC1 is a glycoprotein with extensive O-linked glycosylation of its extracellular domain. MUC1 lines the apical surface of epithelial cells in the lungs, stomach, intestines, eyes and several other organs. Overexpression of MUC1 and in particular its localization to the basal surface of epithelial cells is often associated with colon, breast, ovarian, lung and pancreatic cancers.

The term “ErbB receptor” in particular refers to any and/or all of the members of the ErbB receptor family, in particular of the human ErbB receptor family, especially EGFR/HER1, HER2, HERS and/or HER4. ErbB receptors are receptor tyrosine kinases comprising an extracellular ligand binding domain, a membrane-spanning domain and an intracellular kinase domain. Upon binding of its ligand (e.g. epidermal growth factor (EGF) and transforming growth factor a (TGFα)), the ErbB receptor forms homodimers or heterodimers with other ErbB receptors and its kinase function is activated, resulting in the autophosphorylation of several tyrosines of the intracellular domain. The term “EGFR” according to the present invention in particular refers to the human epidermal growth factor receptor 1, also known as ErbB-1 or HER1.

The term “inhibitor” according to the invention is a molecule that binds to its target, in particular a receptor, and blocks or decreases its activity, in particular its activity in the presence of a ligand. An inhibitor of a receptor may prevent or reduce binding of a ligand to the receptor, formation of active complexes such as receptor dimers, binding of downstream partners to the receptor, modification of the receptor such as phosphorylation, and/or enzyme activity of the receptor such as kinase activity. The inhibitor may be specific for one target, such as EGFR, a specific group of targets such as the members of the ErbB family, or a specific class of targets such as receptor tyrosine kinases.

The term “patient” in particular refers to a human being.

The term “cancer” according to the invention in particular comprises leukemias, seminomas, melanomas, teratomas, lymphomas, neuroblastomas, gliomas, rectal cancer, endometrial cancer, kidney cancer, adrenal cancer, thyroid cancer, blood cancer, skin cancer, cancer of the brain, cervical cancer, intestinal cancer, liver cancer, colon cancer, stomach cancer, intestine cancer, head and neck cancer, gastrointestinal cancer, lymph node cancer, esophagus cancer, colorectal cancer, pancreas cancer, ear, nose and throat (ENT) cancer, breast cancer, prostate cancer, cancer of the uterus, ovarian cancer and lung cancer and the metastases thereof. Examples thereof are lung carcinomas, mamma carcinomas, ovarian carcinomas, prostate carcinomas, colon carcinomas, renal cell carcinomas, cervical carcinomas, or metastases of the cancer types or tumors described above. The term cancer according to the invention also comprises cancer metastases.

By “tumor” is meant a group of cells or tissue that is formed by misregulated cellular proliferation. Tumors may show partial or complete lack of structural organization and functional coordination with the normal tissue, and usually form a distinct mass of tissue, which may be either benign or malignant.

By “metastasis” or “metastases” is meant the spread of cancer cells from its original site (e.g. primary tumor site) to another part of the body. It is not distinguished between singular and plural of “metastasis” except were the context indicates otherwise. The formation of metastasis is a very complex process and normally involves detachment of cancer cells from a primary tumor, entering the body circulation and settling down to grow within normal tissues elsewhere in the body. When tumor cells metastasize, the new tumor is called a secondary or metastatic tumor, and its cells normally resemble those in the original tumor. This means, for example, that, if breast cancer metastasizes to the lungs, the secondary tumor is made up of abnormal breast cells, not of abnormal lung cells. The tumor in the lung is then called metastatic breast cancer, not lung cancer. Metastases can be seen as an embodiment of a neoplastic disease or cancer.

The term “ErbB positive cancer” according to the invention in particular refers to a neoplastic disease, cancer, tumor and/or metastasis wherein cells express one or more members of the ErbB family. The ErbB positive cancer comprises cancer cells which express one or more members of the ErbB family. In certain embodiments, a tumor, metastasis or the like is classified as ErbB positive if a certain percentage of the comprised cells express at least one member of the ErbB family. E.g. in the prior art a tumor is usually classified as being ErbB positive if at least 1% of the tumor cells express at least one member of the ErbB family. If it is referred to a specific member of the ErbB family, such as an EGFR or HER2, then the same applies to this specific ErbB receptor. For example, an EGFR positive cancer comprises cancer cells, in particular a certain percentage of cancer cells such as t least 1%, which express EGFR.

ErbB positive cancers include but are not limited to malignant epithelial tumors, breast cancer, gastric cancer, cancer of the gastrointestinal tract, carcinomas, colon cancer, bladder cancer, urothelial tumors, uterine cancer, esophageal cancer, cancer of the gastroesophageal junction, ovarian cancer, lung cancer, endometrial cancer, kidney cancer, pancreatic cancer, thyroid cancer, colorectal cancer, prostate cancer, cancer of the brain, cervical cancer, intestinal cancer and liver cancer. In certain embodiments, the cancer is a metastasizing cancer. Preferably, the ErbB positive cancer to be treated is selected from colon cancer (including coecum and rectum cancer), lung cancer, breast cancer, ovarian cancer, kidney cancer, gastrointestinal cancer, endometrial cancer, urothelial cancer, and cervical cancer, in particular non-small cell lung cancer (NSCLC) such as squamous non small cell lung cancer (sNSCLC), and non squamous non small cell lung cancer (nsNSCLC).

The term “surgery” according to the invention in particular refers to a surgical removal (resection or ectomy) of tissue comprising all or a part of a tumor, in particular a primary tumor such as a breast tumor, and/or one or more metastases.

An “adjuvant therapy” in particular refers to the treatment of cancer after surgery.

A “neoadjuvant therapy” in particular refers to the treatment of cancer prior to surgery.

A “palliative therapy” in particular refers to a cancer therapy that is given specifically to address symptom management without expecting to significantly reduce the cancer. Palliative care is directed to improving symptoms associated with incurable cancer. The primary objective of palliative care is to improve the quality of the remainder of a patient's life. Pain is one of the common symptoms associated with cancer. Approximately 75% of terminal cancer patients have pain. Pain is a subjective symptom and thus it cannot be measured using technological approaches. The majority of cancer patients experience pain as a result of tumor mass that compresses neighboring nerves, bone, or soft tissues, or from direct nerve injury (neuropathic pain). Pain can occur from affected nerves in the ribs, muscles, and internal structures such as the abdomen (cramping type pain associated with obstruction). Many patients also experience various types of pain as a direct result of follow-up tests, treatments (surgery, radiation, and chemo-therapy) and diagnostic procedures (i.e., biopsy). A therapeutically useful palliative therapy is able to reduce pain.

The term “radiotherapy”, also known as radiation therapy, particularly means the medical use of ionizing radiation to control or kill malignant cells. Radiotherapy may be used in combination with surgery, as adjuvant and/or neoadjuvant therapy, or without surgery, for example to prevent tumor recurrence after surgery or to remove a primary tumor or a metastasis.

The term “pharmaceutical composition” and similar terms particularly refers to a composition suitable for administering to a human, i.e., a composition containing components which are pharmaceutically acceptable. Preferably, a pharmaceutical composition comprises an active compound or a salt thereof together with a carrier, diluent or pharmaceutical excipient such as buffer, or tonicity modifier. According to one embodiment, the pharmaceutical composition does not comprise a preservative.

The terms “antibody composition” and “composition comprising an antibody” are used interchangeably herein if the context does not indicate otherwise. Also the term “antibody” as used herein may in certain embodiments refer to an antibody composition. The antibody composition may be a fluid or solid composition, and also includes lyophilized or reconstituted antibody compositions. Preferably a fluid composition is used, more preferably an aqueous composition. In certain embodiments, it further comprises a solvent such as water, a buffer for adjusting and maintaining the pH value, and optionally further agents for stabilizing the antibody or preventing degradation of the antibody. The antibody composition preferably comprises a reasonable amount of antibodies, in particular at least 1 fmol, preferably at least 1 pmol, at least 1 nmol or at least 1 μmol of the antibody. In certain embodiments, the antibody composition is a pharmaceutical composition.

### DETAILED DESCRIPTION OF THE INVENTION

The present inventors demonstrated that the combined use of an antibody against MUC1 and an inhibitor of a receptor of the ErbB family results in an improved effect in the destruction of tumor cells and hence, in the treatment of cancer. Without being bound to this theory, it is believed that inhibition of the ErbB receptor induces expression of MUC1 in the tumor cells. Thereby, efficacy of the anti-MUC1 antibody is enhanced. Therefore, the present invention pertains to the combined use of an anti-MUC1 antibody and an inhibitor of an ErbB receptor in the treatment of cancer.

The present invention provides an antibody against MUC1 for use in the treatment of cancer in combination with an inhibitor of a receptor of the ErbB family.

Likewise, the present invention provides an inhibitor of a receptor of the ErbB family for use in the treatment of cancer in combination with an antibody against MUC1.

Preferred embodiments of the invention are described subsequently and in the claims to which it is referred.

The Anti-MUC1 Antibody

The antibody against MUC1 or anti-MUC1 antibody is an antibody which is capable of specifically recognizing and binding MUC1.

MUC1 or Mucin-1 is a member of the mucin family and is a glycosylated transmembrane phosphoprotein. The protein is anchored to the apical surface of many epithelial cells by a transmembrane domain. The extracellular domain includes a 20 amino acid variable number tandem repeat (VNTR) domain, with the number of repeats varying from 20 to 120 in different individuals. These repeats are rich in serine, threonine and proline residues which permits heavy O-glycosylation.

In certain embodiments, the anti-MUC1 antibody is directed against an epitope in the extracellular region of MUC1, in particular an epitope in the tandem repeat domain. In certain embodiments, the anti-MUC1 antibody binds MUC1 in a conformation-dependent and/or glycosylation-dependent manner. In particular, the anti-MUC1 antibody binds stronger if said tandem repeats are glycosylated at a threonine residue with N-acetyl galactosamine (Tn), sialyl α2-6 N-acetyl galactosamine (sTn), galactose β1-3 N-acetyl galactosamine (TF) or galactose β1-3 (sialyl α2-6) N-acetyl galactosamine (sTF), preferably with Tn or TF. Preferably, the carbohydrate moiety is bound to the threonine residue by an α-O-glycosidic bond.

In particular embodiments, the antibody is capable of specifically binding to an epitope in the tandem repeat domain of MUC1 which comprises the amino acid sequence PDTR (SEQ ID NO: 19) or PDTRP (SEQ ID NO: 20). The binding to this epitope preferably is glycosylation dependent, as described above, wherein in particular the binding is increased if the carbohydrate moiety described above is attached to the threonine residue of the sequence PDTR or PDTRP, respectively.

In certain embodiments, the anti-MUC1 antibody is directed against a tumor-associated MUC1 epitope (TA-MUC1). A TA-MUC1 epitope in particular refers to an epitope of MUC1 which is present on tumor cells but not on normal cells and/or which is only accessible by antibodies in the host's circulation when present on tumor cells but not when present on normal cells. The epitopes described above, in particular those present in the tandem repeat domain of MUC1, may be tumor-associated MUC1 epitopes. In certain embodiments, the binding of the anti-MUC1 antibody to cells expressing TA-MUC1 epitope is stronger than the binding to cells expressing normal, non-tumor MUC1. Preferably, said binding is at least 1.5-fold stronger, preferably at least 2-fold stronger, at least 5-fold stronger, at least 10-fold stronger or at least 100-fold stronger. In particular, TA-MUC1 is glycosylated with at least one N-acetyl galactosamine (Tn) or galactose β1-3 N-acetyl galactosamine (TF) in its extracellular tandem repeat region. In certain embodiments, the anti-MUC1 antibody specifically binds to this epitope in the extracellular tandem repeat region of TA-MUC1 comprising N-acetyl galactosamine (Tn) or galactose β1-3 N-acetyl galactosamine (TF). Especially, said epitope comprises at least one PDTR or PDTRP (SEQ ID NO: 19 or 20) sequence of the MUC1 tandem repeats and is glycosylated at the threonine of the PDTR or PDTRP (SEQ ID NO: 19 or 20) sequence with N-acetyl galactosamine (Tn) or galactose β1-3 N-acetyl galactosamine (TF), preferably via an α-O-glycosidic bond. For TA-MUC1 binding, the anti-MUC1 antibody preferably specifically binds the glycosylated MUC1 tumor epitope such that the strength of the bond is increased at least by a factor 2, preferably a factor of 4 or a factor of 10, most preferably a factor of 20 in comparison with the bond to the non-glycosylated peptide of identical length and identical peptide sequence.

The anti-MUC1 antibody preferably is a monoclonal antibody. Furthermore, the anti-MUC1 antibody preferably is a human, murine, goat, primate or camel antibody or is derived therefrom. It may be a chimeric or humanized antibody. It may be an antibody of any isotype or subclass thereof, in particular of the IgG, IgM, IgA, IgE or IgD isotype or a subclass thereof such as IgG1. The anti-MUC1 antibody furthermore may be a fragment or derivative of an antibody, for example selected from the group consisting of a Fab fragment, a F(ab)₂ fragment, a Fd fragment, a Fv fragment, a scFv fragment, a (Fv)₂ fragment, and a multibody. Exemplary anti-MUC1 antibodies being an antibody derivative include fusion proteins comprising an antibody or antibody fragment, and mutated antibodies such as mutants lacking the conserved Fc glycosylation site.

The heavy chain variable region comprised in the anti-MUC1 antibody preferably encompasses at least one CDR selected from the group consisting of CDR1 having the amino acid sequence of SEQ ID NO: 1 or 2, CDR2 having the amino acid sequence of SEQ ID NO: 3 or 4, and CDR3 having the amino acid sequence of SEQ ID NO: 5 or 6, preferably at least CDR1 having the amino acid sequence of SEQ ID NO: 1. In particular, it may comprise a set of CDRs wherein CDR1 has the amino acid sequence of SEQ ID NO: 1, CDR2 has the amino acid sequence of SEQ ID NO: 3 and CDR3 has the amino acid sequence of SEQ ID NO: 5, or wherein CDR1 has the amino acid sequence of SEQ ID NO: 2, CDR2 has the amino acid sequence of SEQ ID NO: 4 and CDR3 has the amino acid sequence of SEQ ID NO: 6.

According to one embodiment, the heavy chain variable region comprises the amino acid sequence of SEQ ID NOs: 7, 8 or 9 or an amino acid sequence which is at least 75%, in particular at least 80%, at least 85%, at least 90%, at least 95% or at least 97% homologous or identical to one of said sequences. In certain embodiments, the heavy chain variable region of the anti-MUC1 antibody comprises an amino acid sequence (i) which comprises a set of CDRs wherein CDR1 has the amino acid sequence of SEQ ID NO: 1, CDR2 has the amino acid sequence of SEQ ID NO: 3 and CDR3 has the amino acid sequence of SEQ ID NO: 5, or wherein CDR1 has the amino acid sequence of SEQ ID NO: 2, CDR2 has the amino acid sequence of SEQ ID NO: 4 and CDR3 has the amino acid sequence of SEQ ID NO: 6; and (ii) which is at least 80%, at least 85%, at least 90%, at least 95% identical to any one of SEQ ID NOs: 7, 8 and 9.

The anti-MUC1 antibody may further comprise at least one further complementarity determining region selected from the group consisting of CDR1 having the amino acid sequence of SEQ ID NO: 10 or 11, CDR2 having the amino acid sequence of SEQ ID NO: 12 or 13, and CDR3 having the amino acid sequence of SEQ ID NO: 14 or 15, wherein said at least one further complementarity determining region is preferably present within a light chain variable region. In particular, the anti-MUC1 antibody preferably comprises a set of CDRs wherein the CDRs of the heavy chain variable region have the amino acid sequences of SEQ ID NOs: 1, 3 and 5 and the CDRs of the light chain variable region have the amino acid sequences of SEQ ID NOs: 10, 12 and 14, or wherein the CDRs of the heavy chain variable region have the amino acid sequences of SEQ ID NOs: 2, 4 and 6 and the CDRs of the light chain variable region have the amino acid sequences of SEQ ID NOs: 11, 13 and 15. Said light chain variable region may comprise the amino acid sequence of SEQ ID NO: 16, 17 or 18 or an amino acid sequence which is at least 75%, in particular at least 80%, at least 85%, at least 90%, at least 95% or at least 97% homologous or identical to one of said sequences. In certain embodiments, the light chain variable region of the anti-MUC1 antibody comprises an amino acid sequence (i) which comprises a set of CDRs wherein CDR1 has the amino acid sequence of SEQ ID NO: 10, CDR2 has the amino acid sequence of SEQ ID NO: 12 and CDR3 has the amino acid sequence of SEQ ID NO: 14, or wherein CDR1 has the amino acid sequence of SEQ ID NO: 11, CDR2 has the amino acid sequence of SEQ ID NO: 13 and CDR3 has the amino acid sequence of SEQ ID NO: 15; and (ii) which is at least 80%, at least 85%, at least 90%, at least 95% identical to any one of SEQ ID NOs: 16, 17 and 18.

In particular preferred embodiments, the antibody according to the invention comprises a VH comprising the amino acid sequence of SEQ ID NO: 9 and a VL comprising the amino acid sequence of SEQ ID NO: 18. In a further embodiment, the antibody is derived from an antibody comprising one or more of the sequences described above.

In certain embodiments, the anti-MUC1 antibody is PankoMab in its chimeric or humanized version, or an antibody derived therefrom. The antibody derived from PankoMab in particular binds to the same epitope as PankoMab and/or shows cross-specificity with PankoMab.

In certain embodiment, the anti-MUC1 antibody according to the invention is glycosylated. In particular, the anti-MUC1 antibody has a glycosylation site in the second constant domain of the heavy chain (CH2). An antibody normally has two heavy chains having identical amino acid sequences. Hence, the anti-MUC1 antibody preferably has at least two glycosylation sites, one in each of its two CH2 domains. This glycosylation site in particular is at an amino acid position corresponding to amino acid position 297 of the heavy chain according to the Kabat numbering and has the amino acid sequence motive Asn Xaa Ser/Thr wherein Xaa may be any amino acid except proline. The N-linked glycosylation at Asn297 is conserved in mammalian IgGs as well as in homologous regions of other antibody isotypes. Due to optional additional amino acids which may be present in the variable region or other sequence modifications, the actual position of this conserved glycosylation site may vary in the amino acid sequence of the antibody. Preferably, the glycans attached to the anti-MUC1 antibody are biantennary complex type N-linked carbohydrate structures, preferably comprising at least the following structure:
Asn-GlcNAc-GlcNAc-Man-(Man-GlcNAc)₂
   wherein Asn is the asparagine residue of the polypeptide portion of the antibody; GlcNAc is N-acetylglucosamine and Man is mannose. The terminal GlcNAc residues may further carry a galactose residue, which optionally may carry a sialic acid residue. A further GlcNAc residue (named bisecting GlcNAc) may be attached to the Man nearest to the polypeptide. A fucose may be bound to the GlcNAc attached to the Asn.

In certain embodiments, the anti-MUC1 antibody has a glycosylation pattern at the Fc part which has one or more of the following characteristics:
(i) a relative amount of glycans carrying bisecting N-acetylglucosamine (bisGlcNAc) of at least 1%, in particular at least 2% or at least 5% of the total amount of glycans attached to the Fc part of the anti-MUC1 antibodies in the antibody population;
(ii) a relative amount of glycans carrying at least one sialic acid of 40% or less, in particular 35% or less or 30% or less of the total amount of glycans attached to the Fc part of the anti-MUC1 antibodies in the antibody population; and/or
(iii) a relative amount of glycans carrying at least one galactose residue of at least 30%, in particular at least 40% or at least 50% of the total amount of glycans attached to the Fc part of the anti-MUC1 antibodies in the antibody population.

Preferably, the glycosylation pattern comprises at least two of the features (i), (ii) and (iii) (in particular features (i) and (ii), (i) and (iii), or (ii) and (iii)), and more preferably all of the features (i), (ii) and (iii).

In preferred embodiments, the anti-MUC1 antibody does not comprise N-glycolyl neuraminic acids (NeuGc) or detectable amounts of NeuGc. Furthermore, the anti-MUC1 antibody preferably also does not comprise Galili epitopes (Galα1,3-Gal structures) or detectable amounts of the Galili epitope. In particular, the relative amount of glycans carrying NeuGc and/or Galα1,3-Gal structures is less than 0.1% or even less than 0.02% of the total amount of glycans attached to the Fc part of the anti-MUC1 antibodies in the antibody population.

In particular, the anti-MUC1 antibody has a human glycosylation pattern. Due to these glycosylation properties, foreign immunogenic non-human structures which induce side effects are absent which means that unwanted side effects or disadvantages known to be caused by certain foreign sugar structures such as the immunogenic non-human sialic acids (NeuGc) or the Galili epitope (Gal-Gal structures), both known for rodent production systems, or other structures like immunogenic high-mannose structures as known from e.g. yeast systems are avoided.

In certain embodiments the glycosylation pattern at the Fc part of the anti-MUC1 antibody comprises one or more, preferably all of the following characteristics:
(i) a relative amount of glycans carrying bisecting N-acetylglucosamine (bisGlcNAc) in the range of from 2% to 30% of the total amount of glycans attached to the Fc part of the anti-MUC1 antibodies in the antibody population;
(ii) a relative amount of glycans carrying at least one sialic acid in the range of from 2% to 30% of the total amount of glycans attached to the Fc part of the anti-MUC1 antibodies in the antibody population; and/or
(iii) a relative amount of glycans carrying at least one galactose residue in the range of from 40% to 95% of the total amount of glycans attached to the Fc part of the anti-MUC1 antibodies in the antibody population.

The anti-MUC1 antibody may have a high amount of fucose in the Fc-glycosylation. In these embodiments, the anti-MUC1 antibody has a glycosylation pattern at the Fc part which has a relative amount of glycans carrying fucose of at least 60%, in particular at least 70% or at least 80% of the total amount of glycans attached to the Fc part of the anti-MUC1 antibodies in the antibody population. In alternative embodiments, the anti-MUC1 antibody has a low amount of fucose in the Fc-glycosylation. In these embodiments, the anti-MUC1 antibody has a glycosylation pattern at the Fc part which has a relative amount of glycans carrying fucose of 50% or less, in particular 40% or less or 30% or less of the total amount of glycans attached to the Fc part of the anti-MUC1 antibodies in the antibody population. In particular, the anti-MUC1 antibody has a glycosylation pattern at the Fc part which has a relative amount of glycans carrying fucose of 25% or less, 20% or less or 15% or less of the total amount of glycans attached to the Fc part of the anti-MUC1 antibodies in the antibody population.

The anti-MUC1 antibody is preferably recombinantly produced in a host cell. Hence, the anti-MUC1 antibody in particular is a monoclonal antibody. The host cell used for the production of the anti-MUC1 antibody may be any host cells which can be used for antibody production. Suitable host cells are in particular eukaryotic host cells, especially mammalian host cells. Exemplary host cells include yeast cells such as Pichia pastoris cell lines, insect cells such as SF9 and SF21 cell lines, plant cells, bird cells such as EB66 duck cell lines, rodent cells such as CHO, NSO, SP2/0 and YB2/0 cell lines, and human cells such as HEK293, PER.C6, CAP-T, Mutz-3 and KG1 cell lines.

In certain embodiments, the anti-MUC1 antibody is produced recombinantly in a human blood cell line, in particular in a human myeloid leukemia cell line. Preferred human cell lines which can be used for production of the anti-MUC1 antibody as well as suitable production procedures are described in WO 2008/028686 A2. In a specific embodiment, the anti-MUC1 antibody is obtained by expression in a human myeloid leukemia cell line selected from the group consisting of NM-H9D8, NM-H9D8-E6, NM-H9D8-E6Q12 and GT-5S. These cell lines were deposited under the accession numbers DSM ACC2806 (NM-H9D8; deposited on Sep. 15, 2006), DSM ACC2807 (NM-H9D8-E6; deposited on October 5, 2006), DSM ACC2856 (NM-H9D8-E6Q12; deposited on Aug. 8, 2007), and DSM ACC3078 (GT-5s; deposited on Jul. 28, 2010) according to the requirements of the Budapest Treaty at the Deutsche Sammlung von Mikroorganismen and Zellkulturen (DSMZ), Inhoffenstraße 7B, 38124 Braunschweig (DE) by Glycotope GmbH, Robert-Rossle-Str. 10, 13125 Berlin (DE).

Other suitable cell lines include K562, a human myeloid leukemia cell line present in the American Type Culture Collection (ATCC CCL-243), as well as cell lines derived from the aforementioned.

In certain embodiments, the anti-MUC1 antibody comprises an additional glycosylation site in its Fab fragment, in particular in the heavy chain variable region VH. In preferred embodiments, the anti-MUC1 antibody comprises two heavy chains having identical amino acid sequences and two light chains having identical amino acid sequences. Hence, the anti-MUC1 antibody in certain embodiments comprises two additional glycosylation sites, in particular one in each of its two VH domains.

The glycosylation pattern at the Fab part of the anti-MUC1 antibody may comprise a relative amount of glycans carrying bisGlcNAc of at least 10%, preferably at least 15% or at least 20% of the total amount of glycans attached to the Fab part of the anti-MUC1 antibodies in the antibody population. The amount of bisGlcNAc in the Fab-glycosylation preferably is in the range of from 20% to 85%.

Furthermore, the glycosylation pattern at the Fab part of the anti-MUC1 antibody may comprise a relative amount of glycans carrying at least one galactose residue of at least 60%, preferably at least 70% or at least 80% of the total amount of glycans attached to the Fab part of the anti-MUC1 antibodies in the antibody population.

Furthermore, the glycosylation pattern at the Fab part of the anti-MUC1 antibody may comprise a relative amount of glycans carrying at least one sialic acid residue of at least 40%, preferably at least 50% or at least 60% of the total amount of glycans attached to the Fab part of the anti-MUC1 antibodies in the antibody population.

In particular, these glycosylation characteristics of the Fab part are present in the anti-MUC1 antibody in combination with the glycosylation characteristics of the Fc part described above.

A glycosylation comprising bisGlcNAc, galactose and sialic acid as described above is characteristic for a human glycosylation pattern and can be obtained by expressing the anti-MUC1 antibodies in a human cell line as described above. Sialic acid as mentioned herein preferably refers to N-acetyl neuraminic acid which preferably is coupled to the galactose via an α2,6-, α2,3- or α2,8-bond. According to a preferred embodiment, the anti-MUC1 antibody comprises detectable amounts of a2,6-coupled N-acetyl neuraminic acid (NeuAc).

In another embodiment, the anti-MUC1 antibody is not glycosylated. In specific embodiments, the anti-MUC1 antibody does not comprise a glycosylation site, in particular not the conserved N-glycosylation site in the CH2 domain.

The anti-MUC1 antibody preferably is an IgG antibody, more preferably an IgG1 antibody. It has the ability of specifically binding its target epitope and the ability of binding to Fcγ receptors, in particular to the Fcγ receptor IIIa. The anti-MUC1 antibody is capable of inducing an antibody-dependent cellular cytotoxicity (ADCC) reaction.

In specific embodiments, the anti-MUC1 antibody is provided as conjugate comprising the antibody conjugated to a further agent such as a therapeutically active substance. The anti-MUC1 antibody can be conjugated to one or more further agents. If more than one further agent is present in the conjugate, these further agents may be identical or different, and in particular are all identical. Conjugation of the further agent to the anti-MUC1 antibody can be achieved using any methods known in the art. The further agent may be covalently, in particular by fusion or chemical coupling, or non-covalently attached to the antibody. In certain embodiments, the further agent is covalently attached to the anti-MUC1 antibody, especially via a linker moiety. The linker moiety may be any chemical entity suitable for attaching the further agent to the anti-MUC1 antibody.

The further agent(s) may be coupled to any suitable position of the anti-MUC1 antibody. Coupling may be random or site specific. For example, the further agent(s) may be coupled to specific amino acids such as lysines, methionines or cysteins, or to the N-terminus or the C-terminus of one or more of the poylpeptide chains of the antibody. Furthermore, the further agent(s) may be coupled to amino acid(s) specifically introduced into the amino acid sequence, including artificial amino acids. Moreover, the further agent(s) may be coupled to the carbohydrate chain(s) of the antibody, including the natural carbohydrate chains as well as artificially introduced carbohydrate chains.

The further agent preferably is useful in therapy and/or monitoring of cancer. For example, the further agent may be selected from the group consisting of radionuclides, chemotherapeutic agents, antibodies or antibody fragments, in particular those of different species and/or different specificity than the anti-MUC1 antibody, enzymes, interaction domains, detectable labels, toxins, cytolytic components, immunomodulators, immunoeffectors, MHC class I or class II antigens, and liposomes. A particular preferred further agent is a radionuclide or a cytotoxic agent capable of killing cancer cells, such as a chemotherapeutic agent. In certain preferred embodiments, a chemotherapeutic agent is attached to the anti-MUC1 antibody forming a conjugate.

Specific examples of chemotherapeutic agents that can be conjugated as further agent include alkylating agents such as cisplatin, anti-metabolites, plant alkaloids and terpenoids, vinca alkaloids, podophyllotoxin, taxanes such as taxol, topoisomerase inhibitors such as irinotecan and topotecan, antineoplastics such as doxorubicin or microtubule inhibitors such as auristatins and maytansin/maytansinoids.

The chemotherapeutic agent may in particular be selected from a group consisting of a V-ATPase inhibitor, a pro-apoptotic agent, a Bcl2 inhibitor, an MCL1 inhibitor, a HSP90 inhibitor, an IAP inhibitor, an mTor inhibitor, a microtubule stabilizer, a microtubule destabilizer, an auristatin, a dolastatin, a maytansin, a maytansinoid, amatoxin, a methionine aminopeptidase, an inhibitor of nuclear export of proteins CRM1, a DPPIV inhibitor, proteasome inhibitors, inhibitors of phosphoryl transfer reactions in mitochondria, a protein synthesis inhibitor, a kinase inhibitor, a CDK2 inhibitor, a CDK9 inhibitor, a kinesin inhibitor, an HDAC inhibitor, a topoisomerase I inhibitor, a DNA damaging agent, a DNA alkylating agent, a DNA intercalator, a DNA minor groove binder and a DHFR inhibitor. In specific embodiments, the chemotherapeutic agent attached to the anti-MUC1 antibody is selected from the group consisting of an auristatin, a maytansinoid, a topoisomerase I inhibitor, a DNA damaging agent, a DNA alkylating agent and a DNA minor groove binder.

In some embodiments of the chemotherapeutic agent is a maytansin or maytansinoid. Specific examples of maytansinoids useful for conjugation include maytansinol, N^{2′}-deacetyl-N^{2′}-(3-mercapto-1-oxopropyl)-maytansine (DM1), N^{2′}-deacetyl-N^{2′}-(4-mercapto-1-oxopentyl)-maytansine (DM3), and N^{2′}-deacetyl-N^{2′}-(4-methyl-4-mercapto-1-oxopentyl)-maytansine (DM4). In particular, DM1 or DM4 is attached to the anti-MUC1 antibody. In some embodiments, the chemotherapeutic agent attached to the anti-MUC1 antibody is an auristatin, in particular monomethyl auristatin F (MMAF), monomethyl auristatin E (MMAE) or auristatin T. In some embodiments, the chemotherapeutic agent attached to the anti-MUC1 antibody is a DNA minor groove binder, in particular pyrrolobenzodiazepine (PBD), pyrrolobenzodiazepine dimer (PBD dimer), duocarmycin, duocarmycin-hydroxybenzamide-azaindole (DUBA), seco-duocarmycin-hydroxybenzamide-azaindole (seco-DUBA) or doxorubicin. In some embodiments, the chemotherapeutic agent attached to the anti-MUC1 antibody is a DNA alkylating agent, in particular indolinobenzodiazepine or oxazolidinobenzodiazepine. In some embodiments, the chemotherapeutic agent attached to the anti-MUC1 antibody is a DNA damaging agent, in particular calicheamicin. In some embodiments, the chemotherapeutic agent attached to the anti-MUC1 antibody is a topoisomerase I inhibitor, in particular camptothecin and its derivatives such as 7-ethyl-10-hydroxy-camptothecin (SN-38), (S)-9-dimethylaminomethyl-10-hydroxycamptothecin (topotecan), and (1S,9S)-1-amino-9-ethyl-5-fluoro-1,2,3,9,12,15-hexahydro-9-hydroxy-4-methyl-10H,13H-benzo[de]pyrano[3′,4′:6,7]indolizino[1,2-b]quinoline-10,13-dione (Exatecan (DX-8951f)). Suitable antibody drug conjugates are also described in EP 16 151 774.3 and LU 92659, to which is explicitly referred to herewith.

In further embodiments of the present invention, a binding agent specifically binding to MUC1, in particular to TA-MUC1, is used instead of the anti-MUC1 antibody. The binding agent in particular is conjugated to a therapeutically active substance as described above, especially a cytotoxin. Suitable examples of respective binding agents include anticalins.

The Inhibitor of the Receptor of the ErbB Family

The inhibitor of a receptor of the ErbB family (“ErbB inhibitor”) is an inhibitor which is capable of reducing or eliminating the activity of one or more members of the ErbB receptor family.

As used herein, ErbB family or ErbB receptor family in particular refers to the human ErbB receptors. The human ErbB receptor family, also known as HER receptor family, includes EGFR (HER1, ErbB1), HER2 (ErbB2, Neu), HERS (ErbB3) and HER4 (ErbB4). These members of the ErbB family are transmembrane receptor tyrosine kinases. They comprise an extracellular portion which may be capable of binding to its natural ligand such as EGF and TGF-α. Furthermore, they comprise a transmembrane domain and an intracellular portion. Upon ligand binding, the ErbB receptor dimerizes either as homodimer or as heterodimer with another ErbB family member. Then autophosphorylation of the intracellular portion occurs via the kinase domain. Specific proteins including Ras, P13K, STAT and PLC-γ bind to the phosphorylated receptor and initiate intracellular downstream signaling which in cancer cells may lead to cell survival and proliferation, invasion, metastasis and angiogenesis. In specific embodiments, the receptor of the ErbB family is EGFR or HER2, in particular EGFR.

The inhibitor of a receptor of the ErbB family may inhibit the function of only one member of the ErbB receptor family or it may inhibit the function of two or more, such as all, of the members of the ErbB receptor family. In specific embodiments, the inhibitor inhibits EGFR and/or HER2, in particular EGFR or HER2, especially EGFR. In certain embodiments, the inhibitor is specific for the one or more ErbB receptor family members it inhibits. This means that it does not significantly inhibit other receptors when administered to the human body.

Inhibitors of ErbB receptors may inhibit the function of the receptor by different mechanisms. For example, the inhibitor may prevent binding of the ligand to the receptor, e.g. by blocking the binding site on the receptor, or the inhibitor may prevent dimerization of the receptor, e.g. by blocking the interaction site where the other receptor binds or by sterically hindering the binding to the other receptor, or the inhibitor may prevent phosphorylation of the receptor, e.g. by blocking the kinase domain of the receptor or blocking the phosphorylation site of the receptor.

The inhibitor of a receptor of the ErbB family may be any substance which is suitable for the therapeutic applications described herein. In certain embodiments, the inhibitor is a small molecule, i.e. a compound having a molecular weight of 2,000 Dalton or less, in particular 1,500 Dalton or less or 1,000 Dalton or less. Suitable examples of small molecule inhibitors of a receptor of the ErbB family include Afatinib, Erlotinib, Rociletinib, Lapatinib, Gefintinib, Dacomitinib, Vandetanib, AZD9291, Neratinib, Brigatinib, Icotinib, AZD8931 and Canertinib. These inhibitors in particular inhibit EGFR and/or HER2. Small molecule inhibitors as described herein in particular are inhibitors of the kinase domain of one or more members of the ErbB family.

In other embodiments, the inhibitor is a protein, in particular an antibody. Suitable examples of antibody inhibitors of a receptor of the ErbB family include Cetuximab, Panitumumab, Zalutumumab, Nimotuzumab, Matuzumab and Necitumumab, which are EGFR inhibitors, and Trastuzumab and Pertuzumab, which are HER2 inhibitors. Inhibitors being antibodies in particular are antibodies which are of interfering with or preventing activation of one or more members of the ErbB family, such as EGFR and/or HER2. For example, they may reduce or prevent ligand binding to and/or dimerization of the receptor.

In certain embodiments, the inhibitor of a receptor of the ErbB family is an inhibitor of EGFR, in particular selected from the group consisting of Cetuximab, Erlotinib and Afatinib.

The Cancer to be Treated

The combination therapy using an inhibitor of a receptor of the ErbB family and an anti-MUC1 antibody shows unexpectedly high therapeutic efficacy in cancer patients. In particular, the cancer to be treated is ErbB positive, i.e. it is positive for at least one of the ErbB family members targeted by the inhibitor. In a certain embodiment, the cancer is EGFR positive and the inhibitor is an inhibitor of EGFR. In another embodiment, the cancer is HER2 positive and the inhibitor is an inhibitor of HER2. In another embodiment, the cancer is HER3 positive and the inhibitor is an inhibitor of HER3. In another embodiment, the cancer is HER4 positive and the inhibitor is an inhibitor of HER4.

The cancer may further be positive or negative for MUC1 expression prior to treatment with the ErbB inhibitor. In certain embodiments, the cancer is ErbB positive, in particular EGFR positive, and MUC1 negative. In other embodiments, the cancer is ErbB positive, in particular EGFR positive, and MUC1 positive.

Different forms of cancers including metastases can be treated with the combination therapy according to the invention. The cancer can in particular be selected from the group consisting of colon cancer, lung cancer, ovarian cancer, breast cancer, cervical cancer, endometrial cancer, gastrointestinal cancer, kidney cancer, head and neck cancer and urothelial cancer. Certain examples of cancer that can be treated are colon carcinomas, non-small cell lung carcinomas, squamous cell lung cancer, squamous cell carcinoma of the head and neck, renal cell carcinomas, esophageal adenocarcinomas, gastric adenocarcinomas, gastroesophageal junction adenocarcinomas, endometrial carcinomas or sarcomas and cervical carcinomas, including metastatic forms thereof. Further cancers include non small cell lung cancers such as squamous non small cell lung cancer (sNSCLC), and non squamous non small cell lung cancer (nsNSCLC), in particular adenocarcinoma and large cell carcinoma; small cell lung cancer (SCLC); and gastric cancers such as adenocarcinoma, in particular tubular adenocarcinoma, papillary adenocarcinoma and mucinous adenocarcinoma, signet ring cell carcinoma, adenoid-squamous carcinoma, squamous carcinoma, medullary gastric carcinoma, small cell gastric carcinoma, and non-differentiated gastric carcinoma. The gastric cancer may be located in the pyloric antrum, in the corpus or in the fundus or may be a diffuse gastric cancer in the entire stomach. In certain embodiments, the cancer is a metastasizing cancer. The cancer may include any type of metastases, such as skin metastases, lymph node metastases, lung metastases, liver metastases, peritoneal metastases, pleural metastases and/or brain metastases. In certain embodiments, the cancer has an inflammatory phenotype. In these embodiments, any of the cancer types described above may be an inflammatory cancer.

Preferably, the cancer has a detectable expression of at least one of the ErbB family members targeted by the inhibitor, preferably detectable by immunohistochemistry or in-situ hybridization. It especially includes cells having an ErbB expression which is detectable by immunohistochemistry or in-situ hybridization. In particular, gene amplification of at least one of the ErbB family members targeted by the inhibitor is detectable, preferably by in situ hybridization such as fluorescence in situ hybridization (FISH), silver in situ hybridization (SISH) or chromogen in situ hybridization (CISH). According to certain embodiments, the status of one or more of the ErbB receptor family members, in particular the EGFR status and/or the HER2 status of the patient is determined prior to treatment.

The cancer may or may not have a detectable level of MUC1 or TA-MUC1 expression prior to the treatment. In certain embodiments, the cancer does not have a detectable expression of MUC1 or a detectable amount of TA-MUC1 prior to the treatment. In these embodiments, MUC1 expression or the presence of TA-MUC1 is only induced by the treatment with the ErbB inhibitor. The cancer may be tested on MUC1 or TA-MUC1 level prior to administration of the anti-MUC1 antibody.

In certain embodiments, the cancer includes cells having a KRAS mutation, in particular a mutation resulting in constitutively active K-Ras protein. Examples of respective K-Ras mutants are K-Ras having a mutation at amino acid number 12 such as K-Ras G12V, K-Ras G12D, K-Ras G12C, K-Ras G12S, K-Ras G12A and K-Ras G12R; K-Ras having a mutation at amino acid number 13 such as K-Ras G13D and K-Ras G13R; and K-Ras having a mutation at amino acid number 61 such as K-Ras Q61H, K-Ras Q61K, and K-Ras Q61L. In further embodiments, the cancer is KRAS wildtype, i.e. it does not comprise cells having a KRAS mutation.

In further embodiments, the cancer includes cells having a mutation in the ErbB family member targeted by the inhibitor, in particular an EGFR mutation. Especially the mutation is in the tyrosine kinase domain of the receptor and may result in hyperactivation of the kinase domain. Exemplary mutations of EGFR include G719X, S7681, T790M, L858R, L861Q, exon 19 deletions or insertions, and exon 20 insertions. Similar mutations may be present in one of the other ErbB family members.

The combination therapy according to the present invention using an inhibitor of a receptor of the ErbB family and an anti-MUC1 antibody can also be used in combination with another therapy wherein the cancer is additionally treated with one or more further anti-cancer therapeutic agents such as chemotherapeutic agents or further anti-cancer antibodies to further improve the therapeutic benefit for the patient.

In certain embodiments, the combination therapy according to the invention is used in further combination with one or more anti-cancer agents such as chemotherapeutic agents which are different from the inhibitor of a receptor of the ErbB family and/or one or more further antibodies which are different from the anti-MUC1 antibody. Here, also combination therapies can be used that are established for inhibitors of the ErbB family. The treatment can also be combined with radiotherapy and/or surgery.

Anti-cancer agents that can be used in combination with the inhibitor and the anti-MUC1 antibody may be selected from any chemotherapeutic agent, in particular chemotherapeutic agents known to be effective for treatment of ErbB positive cancers. The type of chemotherapeutic agent also depends on the cancer to be treated. The combination partner may be selected from the group consisting of taxanes such as paclitaxel (Taxol), docetaxel (Taxotere) and SB-T-1214; cyclophosphamide; imatinib; pazopanib; capecitabine; cytarabine; vinorelbine; gemcitabine; anthracyclines such as daunorubicin, doxorubicin, epirubicin, idarubicin, valrubicin and mitoxantrone; aromatase inhibitors such as aminoglutethimide, testolactone (Teslac), anastrozole (Arimidex), letrozole (Femara), exemestane (Aromasin), vorozole (Rivizor), formestane (Lentaron), fadrozole (Afema), 4-hydroxyandrostenedione, 1,4,6-androstatrien-3,17-dione (ATD) and 4-androstene-3,6,17-trione (6-OXO); topoisomerase inhibitors such as irinotecan, topotecan, camptothecin, lamellarin D, etoposide (VP-16), teniposide, doxorubicin, daunorubicin, mitoxantrone, amsacrine, ellipticines, aurintricarboxylic acid and HU-331; platinum based chemotherapeutic agents such as cis-diamminedichloroplatinum(II) (cisplatin), cis-diammine(1,1-cyclobutanedicarboxylato)platinum(II) (carboplatin) and [(1R,2R)-cyclohexane-1,2-diamine](ethanedioato-O,O′)platinum(II) (oxaliplatin), and antimetabolites, in particular antifolates such as methotrexate, pemetrexed, raltitrexed and pralatrexate, pyrimidine analogues such as fluoruracil, gemcitabine, floxuridine, 5-fluorouracil and tegafur-uracil, and purine analogues, selective estrogen receptor modulators and estrogen receptor downregulators.

Furthermore, also therapeutic antibodies can be used as further combination partner. It may be any antibody that is useful in cancer therapy which is different from the anti-MUC1 antibody and the ErbB inhibitor. In particular, the further antibody is approved for cancer treatment by an administration such as the U.S. Food and Drug Administration (FDA), the European Medicines Agency (EMA, formerly EMEA) and the Bundesinstitut fur Arzneimittel and Medizinprodukte (BfArM). Examples of the further antibody that can be used for combination treatment are anti-VEGF antibodies such as bevacizumab (Avastin); anti-CD52 antibodies such as alemtuzumab (Campath); anti-CD30 antibodies such as brentuximab (Adcetris); anti-CD33 antibodies such as gemtuzumab (Mylotarg); and anti-CD20 antibodies such as rituximab (Rituxan, Mabthera), tositumomab (Bexxar) and ibritumomab (Zevalin). Further exemplary antibodies suitable for combination with the cancer therapy described herein include antibodies against antigens selected from the group consisting of CD44, folate receptor a, NeuGc-GM3 ganglioside, DLL-3, RANKL, PTK7, Notch-3, Ephrin A4, insulin-like growth factor receptor 1, activin receptor-like kinase-1, claudin-6, disialoganglioside GD2, endoglin, transmembrane glycoprotein NMB, CD56, tumor-associated calcium signal transducer 2, tissue factor, ectonucleotide pyrophosphatase/phosphodiesterase 3, CD70, P-cadherin, mesothelin, six transmembrane epithelial antigen of the prostate 1 (STEAP1), carcinoembryonic antigen-related cell adhesion molecule 5 (CEACAM5), nectin 4, guanylyl cyclase C, solute carrier family 44 member 4 (SLC44A4), prostate-specific membrane antigen (PSMA), zinc transporter ZIP6 (LIV1 (ZIP6)), SLIT and NTRK-like protein 6 (SLITRK6), trophoblast glycoprotein (TPBG; 5T4), Fyn3, carbonic anhydrase 9, NaPi2b, fibronectin extra-domain B, endothelin receptor ETB, VEGFR2 (CD309), tenascin c, collagen IV and periostin.

The combination therapy described herein can further be combined with checkpoint antibodies, i.e. antibodies blocking or activating immunomodulatory targets. Thereby, inhibitory signals for an immune response can be blocked and/or activating signals can be triggered. Examples of respective targets include CD40, 4-1 BB, OX40, GITR and CD27 as activating targets, CTLA4, PD1, CD80, CD244 and phosphatidylserine as inhibitory targets, and their respective ligands.

In further embodiments, the combination therapy described herein can be combined with the treatment with immunomodulatory compounds such as chemokines, cytokines, growth factors and vaccines. Suitable cytokines in this respect include interferons such as interferon-α, interferon-β and interferon-γ, and interleukins such as interleukin-2, interleukin-6, interleukin-7, interleukin-12, interleukin-15, interleukin-18 and interleukin-21. Suitable growth factors include G-CSF and GM-CSF.

The combination therapy provided herein preferably is for treatment of a primary tumor, a recurrent tumor and/or metastases of such tumors, and in particular is useful for treatment before, during or after surgery and for the prevention or treatment of metastases. The combination therapy provided herein in particular is for the treatment of a patient as adjuvant therapy. In certain embodiments, the combination therapy provided herein is for the treatment of a patient as neoadjuvant therapy or in a combined neoadjuvant-adjuvant therapy. Furthermore, the combination therapy provided herein is for the treatment of a patient as palliative therapy.

The combination therapy provided herein preferably results in inhibition of tumor growth and in particular reduction of tumor size. Furthermore, the occurrence of further metastases is prevented and/or their number is reduced by the treatment. The treatment preferably results in an increase in progression-free survival; and/or an increase in lifespan and thus the overall survival.

Pharmaceutical Compositions and Administration Schemes

The anti-MUC1 antibody and the inhibitor of a receptor of the ErbB family may be present in the same pharmaceutical composition or in separate pharmaceutical compositions. In certain embodiments, the anti-MUC1 antibody and the inhibitor of a receptor of the ErbB family are present in separate pharmaceutical compositions. Said pharmaceutical compositions in particular may be suitable for intravenous injection or oral consumption. They may be aqueous solutions comprising the antibody and/or the inhibitor, or compositions which can be used to prepare compositions suitable for intravenous injection, for example lyophilized compositions. The pharmaceutical compositions may additionally comprise one or more further components, for example solvents, diluents, and/or excipients. The components of the compositions preferably are all pharmaceutically acceptable. The compositions may be solid or fluid compositions, in particular—preferably aqueous—solutions, emulsions, suspensions, lyophilized powders, tablets or pills. Formulations for preparing therapeutic substances such as antibodies and small molecules as pharmaceutical compositions are well-known in the prior art and thus, do not need any detailed description.

The pharmaceutical compositions comprising the anti-MUC1 antibody and/or the ErbB inhibitor may be administered to the patient by any suitable administration route, in particular orally or by intravenous injection. The known dosage regiments of the anti-MUC1 antibody and the ErbB inhibitor may be used in the present combination therapy. However, due to the synergistic effect of the combination therapy, the doses of the anti-MUC1 antibody and/or the ErbB inhibitor may be reduced compared to the known doses. The skilled person is able to determine suitable dosage regiments for the individual therapeutic agents.

The anti-MUC1 antibody and the ErbB inhibitor may be administered concomitantly or sequentially. For example, one of the two therapeutic substances, in particular the ErbB inhibitor, may be administered first and the other may be administered thereafter. The two administration schemes may overlap or the administration of the first therapeutic substance, in particular the ErbB inhibitor, may be stopped before the second therapeutic substance, in particular the anti-MUC1 antibody, is administered. In certain embodiments, the treatment with the anti-MUC1 antibody and the ErbB inhibitor comprises the administration of the ErbB inhibitor prior to the administration of the anti-MUC1 antibody. In particular, the treatment may comprise sequential administration first of the ErbB inhibitor and thereafter of the anti-MUC1 antibody. In specific embodiments, administration of the ErbB inhibitor is started at least 12 hours before administration of the anti-MUC1 antibody is started. In particular, administration of the ErbB inhibitor is started at least 1 day, especially at least 1 and a half days, at least 2 days, at least 4 days, at least 5 days, at least 6 days or at least 7 days, preferably at least 2 days or at least 3 days before administration of the anti-MUC1 antibody is started.

Furthermore, the therapeutic treatment may be composed of two or more administration cycles, wherein in each cycle an administration scheme as described above may be used. In particular, in each cycle the administration of the ErbB inhibitor is started before administration of the anti-MUC1 antibody is started.

In certain embodiments, the combination results in a synergistic effect of the receptor inhibitor and the antibody against MUC1. In particular, the combination therapy as described herein is more effective than a treatment with the anti-MUC1 antibody or the ErbB inhibitor alone.

Further Therapeutic Applications

In a second aspect, the present invention provides an inhibitor of a receptor of the ErbB family for use in the treatment of cancer in combination with an antibody against MUC1. In a further aspect, the present invention provides a kit of parts comprising a pharmaceutical composition comprising an inhibitor of a receptor of the ErbB family and another pharmaceutical composition comprising an antibody against MUC1 for use in the treatment of cancer.

The embodiments, features and examples described herein above also apply to this aspect of the invention. In particular, the inhibitor of a receptor of the ErbB family may be as described herein, the cancer may be as described herein and the antibody against MUC1 may be as described herein.

In particular, the cancer to be treated expresses the receptor of the ErbB family which is inhibited by the inhibitor of a receptor of the ErbB family. The receptor of the ErbB family especially is EGFR or HER2. The ErbB inhibitor may be a small molecule or an antibody, and in particular is selected from the group consisting of Afatinib, Erlotinib and CetuxiMab.

In certain embodiments, the antibody against MUC1 is an antibody against the extracellular repeats of MUC1, in particular an antibody against TA-MUC1, such as PankoMab. The anti-MUC1 antibody in particular may be coupled to a cytotoxin such as an auristatin, maytansin or maytansinoid.

In specific embodiments, the treatment comprises the administration of the ErbB inhibitor prior to the administration of the antibody against MUC1. In particular, administration of the ErbB inhibitor is started at least 1 day, especially at least 2 days before administration of the anti-MUC1 antibody is started. The combination of the ErbB inhibitor and the anti-MUC1 antibody in particular may result in a synergistic effect.

In addition, the present invention likewise provides methods of treatment in accordance with the other aspects of the invention. In particular, the present invention provides a method of treatment of cancer, comprising administering to the patient in need thereof an inhibitor of a receptor of the ErbB family and an antibody against MUC1. All the embodiments and features described herein for the first and second aspect of the invention also likewise apply to the methods of treatment according to the invention.

Numeric ranges described herein are inclusive of the numbers defining the range. The headings provided herein are not limitations of the various aspects or embodiments of this invention which can be read by reference to the specification as a whole. According to one embodiment, subject matter described herein as comprising certain steps in the case of methods or as comprising certain ingredients in the case of compositions refers to subject matter consisting of the respective steps or ingredients. It is preferred to select and combine preferred aspects and embodiments described herein and the specific subject-matter arising from a respective combination of preferred embodiments also belongs to the present disclosure.

### FIGURES

FIG. 1 shows the increase in the number of antibody binding sites per cell for the anti-TA-MUC1 antibody PankoMab on the different cancer cell lines A549 (lung cancer cell line), DU145 (prostate cancer cell line), HSC4 (tongue squamous cell carcinoma cell line) and HCC366 (lung cancer cell line) after treatment with the EGFR inhibitor afatinib, cetuximab (CM/Erbitux) or erlotinib, each used at their respective IC50 concentration. Medium: control experiment without treatment with an EGFR inhibitor.

FIG. 2 shows an exemplary proliferation inhibition assay using PankoMab conjugated to a cytotoxin against tumor cells after pre-treatment with EGFR inhibitors. HSC4 (A, B) and DU145 (C, D) cells were pre-incubated for 3 days with EGFR inhibitor (1 μM for erlotinib (Erlo) and 0.1 μg/ml for Afatinib (Afat)). After 3 days the antigen expression was confirmed by flow cytometry and cells were setup in a proliferation assay for 4 days using PankoMab conjugated to a cytotoxin (SM). Unconjugated PankoMab and isotype matched ADC served as negative control. Proliferation was calculated relative to medium control (A, C). IC50 values calculated from the curves with or without pretreatment are shown in panels B, D.

FIG. 3 shows exemplary internalization assays using PankoMab conjugated to pHrodo red and different target cells after pre-treatment with EGFR inhibitors. DU145 (A, B) and HCC366 (C, D) cells were pre-incubated for 3 days with EGFR inhibitor (1 μM for erlotinib, 0.1 μM for afatinib and 10 μg/ml for Cetuximab (CM)). After 3 days cells were subjected to the internalization assay using PankoMab conjugated to pHrodo red as monitor for internalization to the acidic intracellular compartment. Cells were measured by flow cytometry after 4 hour incubation at 37° C.; incubation at 4° C. served as negative control.

### EXAMPLES

### Example 1

### Upregulation of TA-MUC1 Expression by EGFR Inhibitor Treatment

In the following, it was demonstrated that the treatment of cancer cells with an EGFR inhibitor induces an increased expression of the tumor antigen TA-MUC1.

Cell Culture

The human tumor cell lines DU145 (prostate), MDA-MB-468 (breast), and HCC366 (lung) were routinely cultured using RPMI 1640 supplemented with 10% fetal bovine serum and 1% L-glutamine. A549 (lung), and HSC4 (head&neck) were cultured using DMEM supplemented with 10% fetal bovine serum and 2% L-glutamine (all from Biochrom). All target cells express moderate levels of EGFR (2+), whereas the basal TA-MUC1 levels are 1+ for DU145, A549, 2+ for HSC4 and MDA-MB-468, and 3+ for HCC366.

Flow Cytometry

TA-MUC1 antigen expression of the cells with or without treatment with EGFR inhibitors (anti-EGFR antibody Cetuximab and tyrosine kinase inhibitors erlotinib and afatinib) was assessed at several time points after start of the treatment by quantitative flow cytometric analysis using the QIFIKIT® (Dako) according to the manufacturer's protocol. Briefly, routinely cultured cells were harvested, resuspended in PBS and PankoMab (mIgG1) or appropriate isotype control was added to a final concentration of 100 μg/ml. After 30 min incubation at 4° C. cells were washed three times with wash buffer containing PBS, 1% BSA and 15 mM NaN₃. In parallel setup and calibration beads (included in the QIFIKIT®) were prepared by washing with wash buffer. FITC conjugate (provided with the QIFIKIT®) was diluted 1:50 in PBS and added to cells and beads for 45min. After incubation at 4° C. cells and beads were washed three times and resuspended in wash buffer. Flow cytometric analysis was performed using the BD FACS Canto II. FITC mean fluorescence intensity (MFI) of beads and analyzed cells was determined using BD FACS Diva Software. The antigen-binding capacity (ABC) was calculated according to the manufacturer's instructions and background corrected by subtraction of the ABC of the negative control (mIgG1). Calculations were done using GraphPad Prism software.

Results

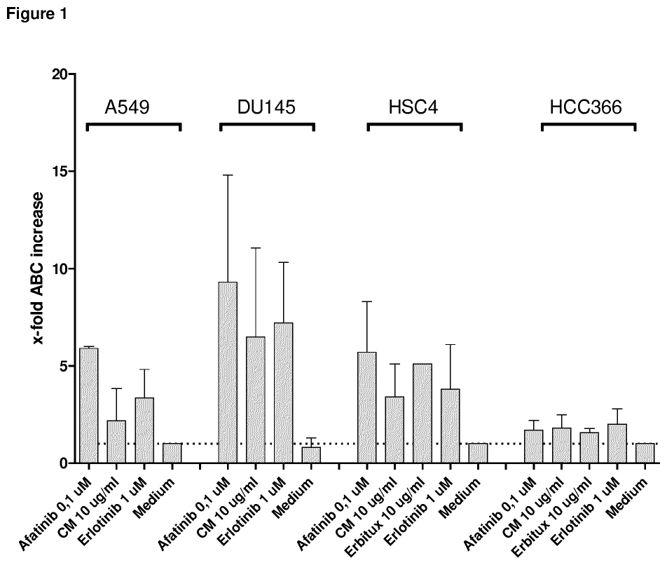

A significant increase in TA-MUC1 expression by the tumor cells upon treatment with the EGFR inhibitors was observed. The increase in the number of antibody binding sites per cell for the anti-TA-MUC1 antibody Pankomab was up to 10-fold (see FIG. 1). In the assay, a significant upregulation of TA-MUC1 was observed starting at day 2 or 3. The effect was concentration dependent, with stronger upregulation by higher inhibitor concentrations. Therefore, it was demonstrated that different EGFR inhibitors are able to increase TA-MUC1 expression on different cancer cell lines.

### Example 2

### Inhibition of Cancer Cell Proliferation by Anti-MUC1 Antibody after EGFR Inhibitor Treatment

In this study, the effect of PankoMab coupled to a cytotoxin on cancer cell proliferation was determined, dependent on the prior treatment of the cancer cells with an EGFR inhibitor.

Cytotoxicity Assay

The cytotoxic potential of a TA-MUC1 targeting antibody drug conjugate (ADC) was investigated using SeeloMab, the ADC format of PankoMab with a microtubule inhibitor as toxin. Therefore, cells were pre-incubated for 3 days with an optimum concentration of EGFR inhibitor. The optimum concentrations were obtained from previous flow cytometric assays and were 1 μM, 0.1 μM or 10 μg/ml for erlotinib, afatinib or cetuximab, respectively. After 3 days the antigen expression was confirmed by flow cytometry and cells were seeded at equal cell density (5000 cells/well) into the wells of a microtiter plate. Cells were incubated with different concentrations of SeeloMab, an isotype matched control ADC and unconjugated PankoMab served as negative control. After 4 days viability of the cells was measured using Celltiter-Glo Luminescent Cell Viability Assay (Promega) and percent proliferation was calculated relative to medium control.

Results

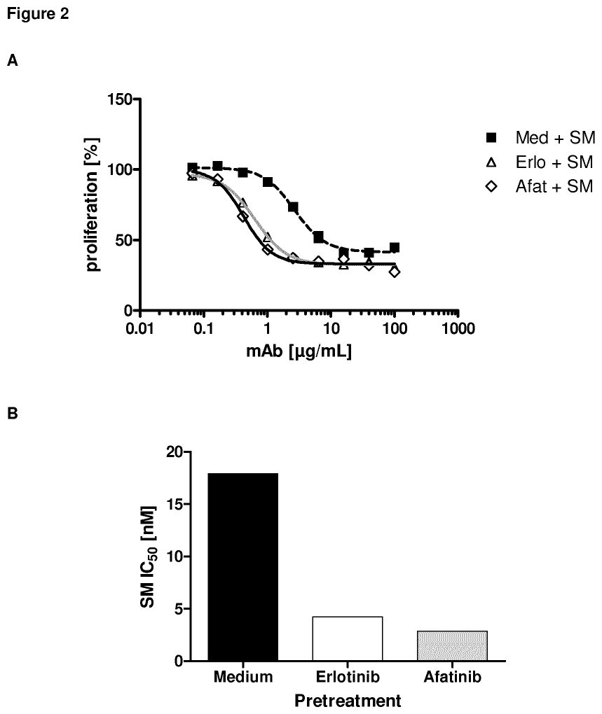

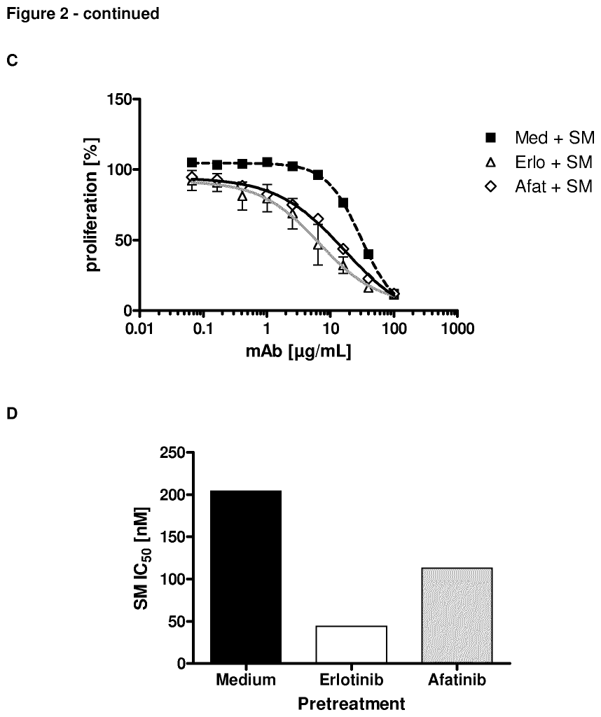

Reduction of proliferation of the cancer cells was observed after addition of the anti-TA-MUC1 ADC surrogate (see FIG. 2). The reduction was significantly stronger when the cancer cells were pretreated with an EGFR inhibitor such as cetuximab or erlotinib. The potency of the combination of EGFR inhibitor and PankoMab-ADC was calculated for the different cancer cell lines from the decrease of the IC50 value (concentration reaching 50% inhibitory effect) of PankoMab-ADC:

TABLE 1
Maximum IC₅₀ ratio of EGFRi pretreated
group compared to non-pretreated group

| Cell line | Cetuximab | Erlotinib | Afatinib |
|---|---|---|---|
| DU145 | 1.3 | 4.2 | 2.3 |
| HSC4 | 1.8 | 9.3 | 13.4 |

The results demonstrated that the pretreatment of the cancer cells with an EGFR inhibitor significantly potentiates the efficacy of PankoMab-ADC.

### Example 3

### Increased Internalization of Anti-MUC1 Antibody by Cancer Cells after EGFR Inhibitor Treatment

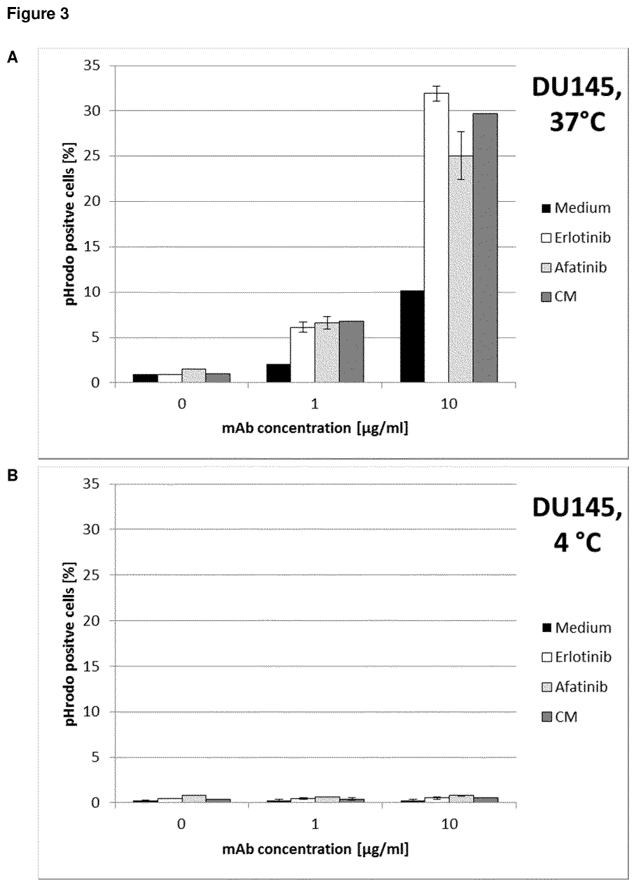

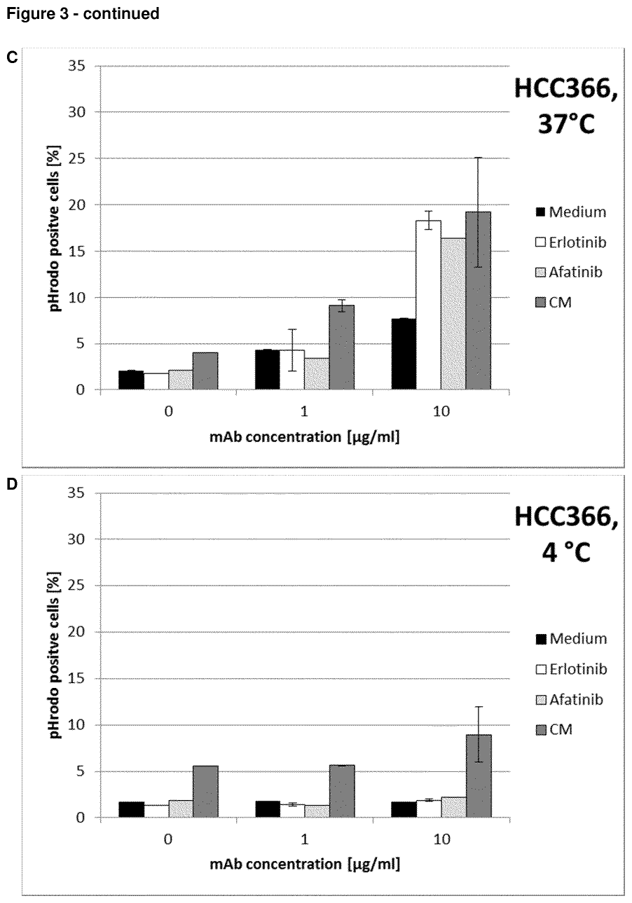

Internalization was measured using PankoMab conjugated to pHrodo red. PHrodo is a pH sensitive fluorescent dye which is non-fluorescent at neutral pH and exhibit increasing signal as the dye is internalized and moved to the acidic lysosomal compartment. Therefore, cells were pre-incubated for 3 days with an optimum concentration of EGFR inhibitor in order to obtain maximum TA-MUC1 expression. The optimum concentrations were obtained from previous flow cytometric assays and were 1 μM, 0.1 μM or 10 μg/mL for erlotinib, afatinib or cetuximab, respectively. After 3 days, cells were harvested and subjected to different antibody concentrations for 1 hour at 4° C. Cells were washed and further incubated for 4 hours at 37° C. to allow for active internalization. Cells incubated at 4° C. served as negative control. After washing and counterstaining of dead cells with 7-AAD, pHrodo fluorescence was measured using the FACS Canto II flow cytometer. FIG. 3 shows a representative assay using DU145 or HCC366 as target cells. For both cell lines concentration dependent increase of pHrodo fluorescent cells was observed at 37° C. whereas only minimal staining was observed at 4° C. indicating active internalization. Cells pretreated with EGFR inhibitors showed 2-3 fold higher percentages of positive cells after 4 hour incubation. This result confirms that the TA-MUC1 upregulation is relevant for PankoMab internalization and possible toxin delivery to tumor target cells.

### Identification of the Deposited Biological Material

The cell lines DSM ACC 2806, DSM ACC 2807, DSM ACC 2856 and DSM ACC 3078 were deposited at the DSMZ—Deutsche Sammlung von Mikroorganismen and Zellkulturen GmbH, InhoffenstraBe 7B, 38124 Braunschweig (DE) by Glycotope GmbH, Robert-Rössle-Str. 10, 13125 Berlin (DE) on the dates indicated in the following table.

| Name of the | Accession | | Date of |
|---|---|---|---|
| Cell Line | Number | Depositor | Deposition |
| NM-H9D8 | DSM ACC 2806 | Glycotope GmbH | Sep. 15, 2006 |
| NM-H9D8-E6 | DSM ACC 2807 | Glycotope GmbH | Oct. 5, 2006 |
| NM-H9D8- | DSM ACC 2856 | Glycotope GmbH | Aug. 8, 2007 |
| E6Q12 | | | |
| GT-5s | DSM ACC 3078 | Glycotope GmbH | Jul. 28, 2010 |

1. A method for treating cancer in a patient, the method comprising administering to the patient an effective amount of an antibody against MUC1 in combination with an inhibitor of an ErbB receptor.

2. The method of claim 1, wherein the cancer expresses the ErbB receptor.

3. The method of claim 1, wherein the cancer is selected from the group consisting of lung cancer, colon cancer, breast cancer and ovarian cancer.

4. The method of claim 1, wherein the antibody against MUC1 is an antibody against the extracellular repeats of MUC1.

5. The method of claim 1, wherein the antibody against MUC1 is an antibody against TA-MUC1.

6. The method of claim 1, wherein the antibody against MUC1 specifically binds to an epitope in the extracellular tandem repeat region of MUC1 comprising N-acetyl galactosamine or galactose β1-3 N-acetyl galactosamine.

7. The method of claim 6, wherein said epitope comprises at least one PDTR or PDTRP (SEQ ID NO: 19 or 20) sequence of the MUC1 tandem repeats and is glycosylated at the threonine of the PDTR or PDTRP (SEQ ID NO: 19 or 20) sequence with N-acetyl galactosamine or galactose 131-3 N-acetyl galactosamine.

8. The method of claim 1, wherein the antibody against MUC1 is PankoMab.

9. The method of claim 1, wherein the anti-MUC1 antibody comprises a set of CDRs wherein the CDRs of the heavy chain variable region have the amino acid sequences of SEQ ID NOs: 1, 3 and 5 and the CDRs of the light chain variable region have the amino acid sequences of SEQ ID NOs: 10, 12 and 14, or wherein the CDRs of the heavy chain variable region have the amino acid sequences of SEQ ID NOs: 2, 4 and 6 and the CDRs of the light chain variable region have the amino acid sequences of SEQ ID NOs: 11, 13 and 15.

10. The method of claim 1, wherein the heavy chain variable region comprises the amino acid sequence of SEQ ID NOs: 7, 8 or 9 or an amino acid sequence which is at least 75% identical to one of said sequences; and wherein the light chain variable region comprises the amino acid sequence of SEQ ID NOs: 16, 17 or 18 or an amino acid sequence which is at least 75% identical to one of said sequences.

11. The method of claim 1, wherein the anti-MUC1 antibody has a glycosylation pattern at the Fc part which has one or more of the following characteristics:
(i) a relative amount of glycans carrying bisecting N-acetylglucosamine (bisGlcNAc) of at least 1% of the total amount of glycans attached to the Fc part of the anti-MUC1 antibodies in the antibody population;
(ii) a relative amount of glycans carrying at least one sialic acid of 40% or less of the total amount of glycans attached to the Fc part of the anti-MUC1 antibodies in the antibody population; and/or
(iii) a relative amount of glycans carrying at least one galactose residue of at least 30% of the total amount of glycans attached to the Fc part of the anti-MUC1 antibodies in the antibody population.

12. The method of claim 1, wherein the antibody against MUC1 is coupled to a cytotoxin.

13. The method of claim 12, wherein the cytotoxin is an auristatin, maytansin or maytansinoid.

14. The method of claim 13, wherein the cytotoxin is monomethyl auristatin E (MMAE).

15. The method of claim 1, wherein the antibody against MUC1 is PankoMab coupled to monomethyl auristatin E (MMAE).

16. The method of claim 1, wherein the ErbB receptor of the ErbB family is EGFR or HER2.

17. (canceled)

18. The method of claim 1, wherein the ErbB inhibitor of an ErbB receptor is a small molecule or an antibody.

19. The method of claim 1, wherein the receptor of the ErbB receptor is EGFR and the ErbB inhibitor is selected from the group consisting of Afatinib, Erlotinib and CetuxiMab.

20. The method of claim 1 comprising administration of the inhibitor prior to the administration of the antibody against MUC1.

21. The method of claim 1, wherein administration of the ErbB inhibitor begins at least 1 day before administration of the anti-MUC1 antibody begins.

22. The method of claim 1, wherein administration of the inhibitor begins at least 2 days before administration of the anti-MUC1 antibody begins.

23. The method of claim 1, wherein the combination results in a synergistic effect.

24. (canceled)

25. (canceled)

26. (canceled)

27. (canceled)


SEQUENCE LISTING (20 sequences)

SEQ ID NO: 1 - protein, 5 aa, Artificial
  CDR H1
NYWMN

SEQ ID NO: 2 - protein, 5 aa, Artificial
  CDR H1
DAWMD

SEQ ID NO: 3 - protein, 19 aa, Artificial
  CDR H2
EIRLKSNNYTTHYAESVKG

SEQ ID NO: 4 - protein, 19 aa, Artificial
  CDR H2
EIRSKANNHATYYAESVKG

SEQ ID NO: 5 - protein, 6 aa, Artificial
  CDR H3
HYYFDY

SEQ ID NO: 6 - protein, 7 aa, Artificial
  CDR H3
GGYGFDY

SEQ ID NO: 7 - protein, 117 aa, Artificial Sequence
  heavy chain variable region
EVKLVESGGGLVQPGGSMKLSCVASGFTFSNYWMNWVRQSPEKGLEWVAEIRLKSNNYTTHYAESVKGRFTISRDDSKSSVSLQMNNLRVEDTGIYYCTRHYYFDYWGQGTTLTVSS

SEQ ID NO: 8 - protein, 117 aa, Artificial
  heavy chain variable region
  MISC_FEATURE (3)..(3): Xaa is Gln or Lys
  MISC_FEATURE (18)..(18): Xaa is Leu or Met
  MISC_FEATURE (23)..(23): Xaa is Ala or Val
  MISC_FEATURE (40)..(40): Xaa is Ala or Ser
  MISC_FEATURE (42)..(42): Xaa is Gly or Glu
  MISC_FEATURE (49)..(49): Xaa is Gly or Ala
  MISC_FEATURE (79)..(79): Xaa is Asn or Ser
  MISC_FEATURE (81)..(81): Xaa is Leu or Val
  MISC_FEATURE (99)..(99): Xaa is Thr or Ala
  MISC_FEATURE (113)..(113): Xaa is Val or Leu
EVXLVESGGGLVQPGGSXRLSCXASGFPFSNYWMNWVRQXPXKGLEWVXEIRLKSNNYTTHYAESVKGRFTISRDDSKXSXYLQMNSLKTEDTAVYYCXRHYYFDYWGQGTLXTVSS

SEQ ID NO: 9 - protein, 117 aa, Artificial
  heavy chain variable region
EVQLVESGGGLVQPGGSMRLSCVASGFPFSNYWMNWVRQAPGKGLEWVGEIRLKSNNYTTHYAESVKGRFTISRDDSKNSLYLQMNSLKTEDTAVYYCTRHYYFDYWGQGTLVTVSS

SEQ ID NO: 10 - protein, 16 aa, Artificial
  CDR L1
RSSKSLLHSNGITYFF

SEQ ID NO: 11 - protein, 16 aa, Artificial
  CDR L1
RSSQSIVHSNGNTYLE

SEQ ID NO: 12 - protein, 7 aa, Artificial
  CDR L2
QMSNLAS

SEQ ID NO: 13 - protein, 7 aa, Artificial
  CDR L2
KVSNRFS

SEQ ID NO: 14 - protein, 9 aa, Artificial
  CDR L3
AQNLELPPT

SEQ ID NO: 15 - protein, 9 aa, Artificial
  CDR L3
FQGSHVPLT

SEQ ID NO: 16 - protein, 114 aa, Artificial Sequence
  light chain variable region
DIVMTQAAFSNPVTLGTSASISCRSSKSLLHSNGITYFFWYLQKPGLSPQLLIYQMSNLASGVPDRFSSSGSGTDFTLRISRVEAEDVGVYYCAQNLELPPTFGGGTKLEIKRA

SEQ ID NO: 17 - protein, 114 aa, Artificial
  light chain variable region
  MISC_FEATURE (11)..(11): Xaa is Leu or Asn
  MISC_FEATURE (17)..(17): Xaa is Glu or Asp
  MISC_FEATURE (79)..(79): Xaa is Lys or Arg
  MISC_FEATURE (105)..(105): Xaa is Gln or Gly
DIVMTQSPLSXPVTPGXPASISCRSSKSLLHSNGITYFFWYLQKPGQSPQLLIYQMSNLASGVPDRFSGSGSGTDFTLXISRVEAEDVGVYYCAQNLELPPTFGXGTKVEIKRA

SEQ ID NO: 18 - protein, 114 aa, Artificial
  light chain vatiable region
DIVMTQSPLSNPVTPGEPASISCRSSKSLLHSNGITYFFWYLQKPGQSPQLLIYQMSNLASGVPDRFSGSGSGTDFTLRISRVEAEDVGVYYCAQNLELPPTFGQGTKVEIKRA

SEQ ID NO: 19 - protein, 4 aa, Artificial Sequence
  epitope
PDTR

SEQ ID NO: 20 - protein, 5 aa, Artificial Sequence
  epitope
PDTRP